(19) United States
(12) Plant Patent Application Publication
(10) Pub. No.: US 2007/0136902 P1
(43) Pub. Date: Jun. 14, 2007

(54) Miniature rose plant name 'Poulpar031'

(76) Inventor: Mogens N. Olesen, Fredensborg (DK)

Correspondence Address: POULSEN ROSER APS, 620 S. FRONT STREET, CENTRAL POINT, OR, 97502, US

(21) Appl. No.: 11/298,192

(22) Filed: Dec. 9, 2005

(51) Int. Cl. A01H 5/00

(52) U.S. Cl. PLT/119

(57) ABSTRACT

A new miniature rose plant that has abundant, orange flowers and attractive foliage. The variety successfully propagates from softwood cuttings and is suitable for year-round production in commercial glasshouses. This new and distinct variety has shown to be uniform and stable in the resulting generations from asexual propagation.

### BOTANICAL CLASSIFICATION

Rosa hybrid

### VARIETY DENOMINATION

‘Poulpar031’

### SUMMARY OF THE INVENTION

The present invention constitutes a new and distinct variety of miniature rose plant which originated from a controlled crossing between the female parent, an unnamed seedling, and the male pollen parent, an unnamed seedling. The two parents were crossed and the resulting seeds were planted in a controlled environment. The new variety is named ‘Poulpar031’.

The new rose may be distinguished from the seed parent by the following combination of characteristics:
1. ‘Poulpar031’ has 35 flower petals. The seed parent has about 50 petals.
2. ‘Poulpar031’ has orange flowers, while the seed parent has pink flowers.
3. ‘Poulpar031’ has smaller flowers than the seed parent.

The new variety may be distinguished from the pollen parent by the following combination of characteristics:
1. ‘Poulpar031’ has 35 flower petals. The pollen parent has approximately 45 to 50 petals.
2. ‘Poulpar031’ has continuous blooming habit; whereas, the unnamed pollen parent has recurrent blooming habit.
3. ‘Poulpar031’ has an orange flowers, while the pollen parent has yellow flowers.

The objective of the hybridization of this rose variety for commercial culture was to create a new and distinct variety with unique qualities, such as:
1. Uniform and abundant orange flowers;
2. Vigorous and compact growth;
3. Year-round flowering under glasshouse conditions;
4. Suitability for production from softwood cuttings in pots;
5. Durable flowers and foliage which make a variety suitable for distribution in the floral industry.

This combination of qualities is not present in previously available commercial cultivars of this type, known to the inventor, and distinguish ‘Poulpar031’ from all other varieties of which we are aware.

As part of the rose development program, Mogens N. Olesen germinated the seeds from the aforementioned hybridization and conducted evaluations on the resulting seedlings in a controlled environment in Fredensborg, Denmark.

‘Poulpar031’ was selected by the inventor as a single plant from the progeny of the hybridization in 2001.

Asexual reproduction of ‘Poulpar031’ by vegetative cuttings and traditional budding was first done by Mogens N. Olesen in the nursery in Fredensborg, Denmark in April 2002. This initial and other subsequent propagations conducted in controlled environments have demonstrated that the characteristics of ‘Poulpar031’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems of ‘Poulpar031’. Specifically illustrated in the drawing:

FIG. 1.1; Stem showing flowers at various stages of development, branching, and the attachment of leaves;

FIG. 1.2; Flower bud closed, partially open, and open;

FIG. 1.3; Flower petals, detached;

FIG. 1.4; Sepals, receptacle, and pedicel;

FIG. 1.5; Stem as well as a bare stem exhibiting thorns;

FIG. 1.6; Leaves.

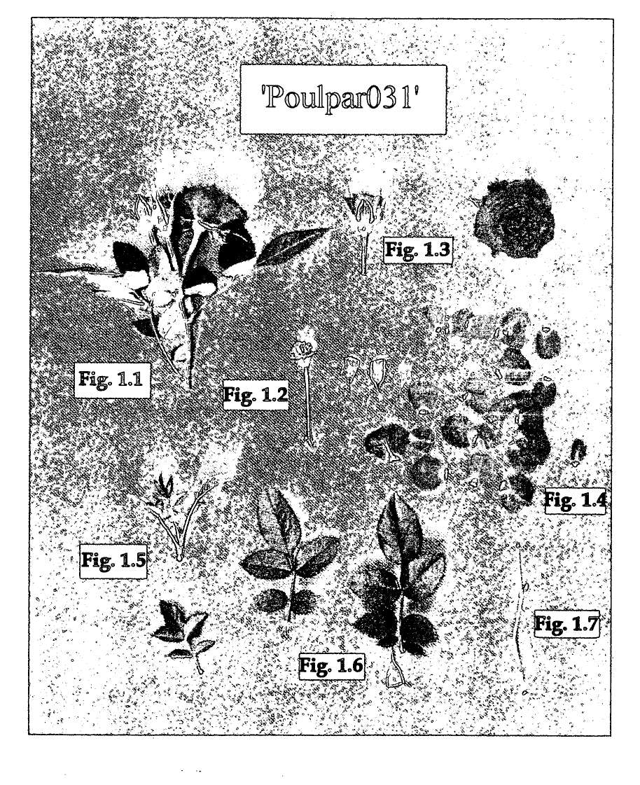

### DETAILED DESCRIPTION OF THE VARIETY

The following is a description of ‘Poulpar031’, as observed in its growth in glasshouses in Odense, Denmark. Observed plants are 4 months of age. Color references are made using The Royal Horticultural Society (London, England) Colour Chart, 2001, except where common terms of color are used.

For a comparison, several physical characteristics of the rose variety ‘Poulnil’, a rose variety from the same inventor described and illustrated in U.S. Plant Pat. No. 13,301, are compared to ‘Poulpar031’ in Chart 1.

CHART 1

| | ‘Poulpar031’ | ‘Poulnil’ |
|---|---|---|
| Petal count | 35 | 16 to 19 |
| Bud color as sepals | Red Group 40A. | Orange-Red Group 31A/B. |
| unfold. | | |
| Flower diameter. | 35 to 40 mm. | 65 to 75 mm. |

Flower and flower bud:
Blooming habit: Continuous.
Flower bud:
   Size.—Upon opening, 18 mm in length from base of receptacle to end of bud. 10 mm in diameter.
   Bud form.—Urceolate.
   Bud color.—As sepals unfold, Red Group 40A.
   Sepals.—Upper Surface: Color: Green Group 138B at the sepal base. Surface: Weak pubescence observed. Lower Surface: Color: Yellow-Green Group 146A. Anthocyanic pigments Greyed-Red 176A at apex of foliaceous appendages. Texture: Smooth with few stipitate glands present. Shape: Apex: Cirrhose. Base: Flat at union with receptacle. Margins: Margins have strong to medium foliaceous appendages and stipitate glands. Size: Average sepal measurements are 25 mm (l) by 7 mm (w).
   Receptacle.—Surface Texture: Smooth. Shape: Urn-shaped. Size: 6 mm (h)×8 mm (w). Color: Green Group 144B. Anthocyanin: None observed.
   Peduncle.—Surface: Smooth. Length: 35 to 38 mm. Diameter: 3 mm on average. Color: Yellow-Green Group 144A to 144B. Strength: Strong.
   Borne.—Singularly and in clusters of 5 flower buds per stem. Inflorescence type is a corymb.
Flower bloom:
   Fragrance.—None to very light floral scent.
   Duration.—As a pot plant, flowers last 18 to 25 days. Petals do not fall cleanly away from plant after flowers have matured.
   Size.—Average flower diameter is 35 to 40 mm when open. Depth is normally 15 mm.
   Form.—Calathiform rosette with a tight center.
   Shape of flower when viewed from the side.—Upon opening, upper part: Flat. Upon opening, lower part: Flat. Open flower, upper part: Flat to flattened convex. Open flower, lower part: Concave.
   Petalage.—Normally 35 petals with 3 to 5 petaloids.
Flower color:
   Upon opening, petals.—Outermost petals: Upper Surface: Red Group 40A with occasional streaks of Yellow-Green Group 145D. Lower Surface: Red Group 43B to 43C. Innermost petals: Upper Surface: Red Group 40A. Lower Surface: Red Group 43B to 43C.
   Upon opening, basal petal spots.—Upper Surface: Yellow Group 4D. Lower Surface: Yellow Group 4D.
   After opening, petals.—Outermost petals: Upper Surface: Red Group 40A with occasional streaks of Yellow-Green Group 145D. Lower Surface: Red Group 43B to 43C. Innermost petals: Upper Surface: Red Group 40A. Lower Surface: Red Group 43B to 43C.
   After opening, basal petal spots.—Outermost petals: Upper Surface: Yellow Group 4D. Lower Surface: Yellow Group 4D.
General tonality: On open flower Red Group 40A. No change in the general tonality as the flower mature.
Petals:
   Petal reflex.—Moderately reflexed.
   Petal margin.—Entire and uniform with a point at the center of the apex. Weak undulations of margin observed.
   Shape.—Generally round to broadly elliptical. Base: Acute. Apex: Cuspidate.
   Size.—Outer petals: 25 mm (l)×25 mm (w). Inner petals: 16 mm (l)×16 mm (w).
   Texture.—Smooth.
   Thickness.—Average.
   Arrangement.—Not formal.
Petaloids:
   Size.—12 mm (l)×5 mm (w).
   Shape.—Narrow elliptical.
   Color.—Upper surface is Red Group 40A with occasional streaks of Yellow-Green Group 145D. Lower surface is Red Group 43B to 43C.
Reproductive organs:
   Pollen.—None observed.
   Anthers.—Size: Approximately 2 mm long. Color: Yellow Group 13A. Quantity: 65.
   Filaments.—Color: Yellow Group 4B. Length: Normally 2 to 3 mm.
   Pistils.—Length: Approximately 4 mm. Quantity: Average 40.
   Stigmas.—Superior relative to the length of the filaments and the height of the anthers. Color: Yellow-Green Group 150D to Yellow-White Group 158C.
   Styles.—Color: Yellow-Green Group 150D.
   Seed formation.—Not observed.
Plant:
Plant growth: Bushy and very compact. When grown as a 10 cm pot plant on its own roots, the average height of the plant itself is 15 to 20 cm and the average width is 15 cm.
Stems:
   Color.—Young wood: Yellow-Green Group 145B. Older wood: Yellow-Green Group 146A.
   Diameter.—2.5 mm on average.
   Internodal distance.—Normally 25 mm.
   Length of stems.—On average canes are 13 cm from the base of the stem to the flowering portion.
   Surface texture.—Young wood: Smooth. Older wood: Smooth.
Prickles:
   Incidence.—4 prickles per 10 cm of stem.
   Color.—Juvenile growth exhibits prickles which are Greyed-Red Group 182A.
   Shape.—Upper side: Flat. Lower side: Flat.
Plant foliage: Normal number of leaflets on normal leaves in middle of the stem: 5 leaflets.
   Compound leaf size.—75 mm (l)×50 mm (w).
   Quantity.—4 leaves per 10 cm of stem.
   Color.—Juvenile foliage: Upper surface of leaflets is Yellow-Green Group 146A. Lower surface is Yellow-Green Group 145A with generalized anthocyanin the color Greyed-Orange Group 174A. Mature foliage: Upper surface is Yellow-Green Group 146A to 147A. Upper leaflet margins exhibit anthocyanin Greyed-Orange Group 176A on occasion. The lower leaflet surface is Yellow-Green Group 147B.
Plant leaves and leaflets:
   Stipules.—Size: 10 to 12 mm. Shape: Linear, slightly broad based with outward extending apecies. Margins: Finely serrated with few stipitate glands. Color: Yellow-Green Group 144A.
   Petiole.—Length: Normally 15 mm. Diameter: Normally 2 mm. Color: Upper surface is Yellow-Green Group 144A. At point of leaflet attachment, Greyed-Orange Group 174B. The lower surface is Yellow-Green Group 144A. Underneath: Few small prickles observed. Few stipitate glands observed.
   Rachis.—Size: 25 mm on average. Color: The upper surface is Yellow-Green Group 144A. At point of leaflet attachment, Greyed-Orange Group 174B. The lower surface is Yellow-Green Group 144A. Underneath: Few small prickles observed. Few stipitate glands observed.
   Leaflet.—Size: 35 to 48 mm (l)×20 to 25 mm (w). Edge: Doubly serrated. General Shape: Elliptical to ovate. Apex Shape: Pointed. Base Shape: Obtuse. Texture: Smooth. Thickness: Average to thin. Arrangement: Odd pinnate. Venation: Reticulate. Leaf Gloss: Matte finish.
Disease resistance: Average resistance to powdery and downy mildews, black spot, and Botrytis under normal growing conditions in Fredensborg, Denmark.

1. A new and distinct variety of rose plant of the miniature class named ‘Poulpar031’ substantially as illustrated and described herein, due to its abundant, orange flowers, vigorous growth, compact habit, suitability for production from softwood cuttings in pots, and durable flowers and foliage that make the variety suitable for distribution in the floral industry
